(19) United States
(12) Patent Application Publication
(10) Pub. No.: US 2023/0355315 A1
(43) Pub. Date: Nov. 9, 2023

(54) SURGICAL NAVIGATION SYSTEM AND APPLICATIONS THEREOF

(71) Applicant: 8Chili, Inc., Rocklin, CA (US)

(72) Inventors: Aravind Kumar UPADHYAYA, Bangalore, Karnataka (IN); Abhishek Settigere VENKATARAM, Bangalore, Karnataka (IN); Sanidhya RASIWASIA, Assam (IN); Ajay HERUR, Bangalore, Karnataka (IN)

(73) Assignee: 8Chili, Inc., Rocklin, CA (US)

(21) Appl. No.: 17/905,177

(22) PCT Filed: Feb. 28, 2021
(86) PCT No.: PCT/US2021/020168

§ 371 Date: Aug. 28, 2022

Related U.S. Application Data
(60) Provisional application No. 62/983,405, filed on Feb. 28, 2020.
(60) Provisional application No. 62/983,427, filed on Feb. 28, 2020.
(60) Provisional application No. 62/983,432, filed on Feb. 28, 2020.

(51) Int. Cl. A61B 34/20; A61B 34/10; A61B 90/50; A61B 90/00

(52) U.S. Cl. CPC A61B 34/20; A61B 34/10; A61B 90/50; A61B 90/361; A61B 2034/107; A61B 2090/502

(57) ABSTRACT

Aspects of the disclosure are presented for a multifunctional platform that is configured for surgical navigation and is portable for use in different locations. The system includes a hardware component and a software component. The hardware component may include a portable or wearable device that can obtain multiple types of input data that can be used in remote visualization of a surgical setting. The hardware may include a headset with various types of cameras, such as a position camera and a visual camera for capturing 2D and 3D data, and circuitry for fusing or overlaying the 2D and 3D images together. In other cases, the hardware may include a bar attachment to a mobile device, such as a smart pad, with multiple camera sensors built in. In some embodiments, the hardware also includes a portable navigation system that can fulfill the functions of both surgical navigation and a surgical microscope.

### CROSS REFERENCES TO RELATED APPLICATIONS

This application claims the benefits of U.S. Provisional Application 62/983,405, filed Feb. 28, 2020, and titled, “MULTIFUNCTIONAL SURGICAL NAVIGATION APPARATUS OR PLATFORM AND APPLICATIONS THEREOF”; U.S. Provisional Application No. 62/983,427, filed Feb. 28, 2020, and titled “SURGICAL NAVIGATION SYSTEM SOFTWARE AND APPLICATIONS THEREOF”; and U.S. Provisional Application 62/983,432, filed Feb. 28, 2020, and titled, “SURGICAL NAVIGATION SYSTEM AND APPLICATIONS THEREOF”; the disclosures of which are incorporated herein by reference in their entireties and for all purposes.

### BACKGROUND

Surgical Navigation and Surgical Microscope machines are two bulky devices mostly independent of each other but are both currently used in many surgeries. It takes surgeons time to shift between these devices during neuro surgeries. Surgical Navigation machines take an average 10-15% of the operating room space and Surgical Microscopes take on an average 15-20% of the space. FIG. 1 is an example of these types of machines that can be very useful during a surgical procedure, but are extremely cumbersome to use.

Both of these devices are portable only in the sense that they are heavy carts with wheels, They easily weigh upwards of 200 kg., so it is simply not practical to have these used outside of an operating room, such as in the emergency or surgical ICU. Once these devices are in the operating room, they tend to stay there for their lifetime. If they are to move in and around the operating room, assistance is required from medical personnel because of their weight.

In the operating room, the surgeons usually tend to use one device at a time, and then they have to keep moving back and forth between either the Surgical Microscope or the Surgical Navigation, depending on their function during the procedure. This back and forth creates discomfort to the surgeon and also increases surgical time creating system inefficiencies and also higher anesthesia because longer surgical time means longer anesthesia.

Procedural physicians, such as surgeons and interventional medical specialists, have a high risk for work-related injuries, such as musculoskeletal disorders (MSDs). This is due to long work hours involving repetitive movements, static and awkward postures, and challenges with instrument design, especially given the rapid rate of innovation in the setting of a diversifying workforce.

Ergonomists have described the surgeon's work environment and working conditions as equal to, if not at times harsher than, those of certain industrial workers.

This observation is consistent with studies demonstrating higher prevalence estimates of work-related injuries among at-risk physicians compared with the general population and even labor-intensive occupations, such as coal miners, manufacturing laborers, and physical therapists.

Although great strides have been made in industrial ergonomics to reduce the burden of disease, medicine has proven to be a unique challenge and the lack of intervention in this group is now becoming apparent.

The surgeons also have limitations in using surgical instruments with navigation systems because there is a line of sight issue with traditional systems. If the surgical instrument gets blocked for whatever reason, then the navigation stops. The optical tracking camera typically needs to have a direct line of sight to the surgical instruments.

The standard way of doing the image guided surgery is not by looking at the surgical site but by looking at the navigation screen and then moving the surgical instruments to the target location by looking at the screen based 2D display—this requires extreme careful maneuverability that only comes from a lot of surgical experience.

The existing navigation systems provide 2D image views from 3 angles (Transverse plane, Sagittal Plane and Coronal Plane). The surgeon then correlates all of this to a 3D point in the patient organ. The surgeon then has a daunting task of mind mapping this 2D info to 3D info from their experience. Hence, this process is inconsistent because a proper 3D visualization is currently unavailable.

There are manual errors that can seep in when doing co-registration. The co-registration process is selecting correlating points first on the software then on the patient. It is common to have errors in point selection because of the human element.

The current surgical navigation and microscope systems are stuck inside the operating room and hence takes additional OR time in setting up due to the need for a surgical plan and pre-op planning discussion.

The current systems perform single functions—surgical navigation, surgical microscopy, Fluorescence visualization, Raman Spectroscopy, Confocal microscopy. There is no one device that can do all this to greatly increase the surgeon's efficiency of not having to switch between devices.

The interventional suite or surgical ICU rooms do not have access to these navigation devices for some of their procedures that can greatly increase patient outcome and satisfaction like epidural injections of the spine and targeted injections to the liver.

It would therefore be desirable to provide a more mobile navigation system to aid in multiple medical procedure contexts. It would also be desirable to allow for a user, such as a surgeon, to be able to more easily perform their tasks remotely, through the use of an improved navigation system interface.

### BRIEF SUMMARY

Aspects of the disclosure are presented for a multifunctional platform that is configured for surgical navigation, surgical microscopy, loupe, and/or fluorescence visualization, that is portable for use in different locations. In some implementations, the platform weighs under 130 pounds. The system includes a hardware component and a software component. The hardware component may include a portable or wearable device that can obtain multiple types of input data that can be used in remote visualization of a surgical setting. In some cases, the hardware includes a headset with various types of cameras, such as a position camera and a visual camera for capturing 2D and 3D data, and circuitry for fusing or overlaying the 2D and 3D images together. In other cases, the hardware may include a bar attachment to a mobile device, such as a smart pad or laptop, with multiple camera sensors built in. In some embodiments, the hardware also includes a portable navigation system that can fulfill the functions of both surgical navigation and a surgical microscope.

The software of the present disclosure may include modules for processing the input data received from one or more of the hardware components and converting the data into an augmented reality (AR) or virtual reality (VR) experience that a remote user can utilize for performing at least some of a surgical procedure.

In some embodiments, an augmented reality device is presented. The AR device may include: a housing; a depth camera coupled to the housing and configured to provide image data with a 3-dimensional component; a visual camera coupled to the housing and configured to provide extra-sensory image data that a human user cannot see naturally; and an overlay display component configured to receive at least two sets of image data and overlay both of the at least two sets of image data onto a common point of reference in a user's field of view.

In some embodiments, the augmented reality device further includes a headset configured to support the housing.

In some embodiments of the augmented reality device, the depth camera and the visual camera are positioned on the headset such that the user's field of view coincides with the both the fields of view of the depth camera and the visual camera.

In some embodiments of the augmented reality device, the overly display component is positioned over the user's field of view as the user wears the headset.

In some embodiments, the augmented reality device further includes a bar attachment configured to attach to a mobile device.

In some embodiments of the augmented reality, the overlay display component utilizes a visual display of the mobile device.

In some embodiments, a system for surgical navigation is presented. The system may include: a first augmented reality (AR) device positioned in a local geographic location; a second augmented reality device positioned in a remote geographic location and wired or wirelessly coupled to the first AR device; and a software system coupled to both the first AR device and the second AR device and configured to: process real-time image data produced by the first AR device; access fixed medical image data recorded previously; and cause the second AR device to display the real-time image data and the fixed medical image data superimposed over the real-time image data.

In some embodiments of the system, the first AR device is configured to identify a fixed reference marker in the field of view and transmit image data about the fixed reference marker to the second AR device.

In some embodiments, of the system, the software system is configured to orient the fixed medical image data to the real-time image data using the image data about the fixed reference marker.

In some embodiments of the system, the fixed medical image data comprises 2D and 3D image data.

In some embodiments of the system, the software system is configured to cause display of both 2D and 3D image data about the patient superimposed over the real-time image data, simultaneously.

In some embodiments of the system, the superimposed 2D and 3D data over the real-time image data represents one or more views of physical content within or inside an object of the real-time image data.

In some embodiments, a method of augmented reality (AR) for fusing digital image data of an object to a real-time view of the object is presented. The method may include: accessing, in real-time, a view of the object; accessing the digital image data of the object, the digital image data of the object previously captured and stored as one or more static digital images of the object; and performing a fusion technique that affixes the digital image data to the view of the object in real-time, using an augmented reality display screen, such that the digital image data stays affixed to the view of the object in real-time as the view of the object changes in position or orientation within the augmented reality display screen.

In some embodiments of the method, the digital image data comprises 3D digital image data of the object.

In some embodiments of the method, the digital image data comprises 2D digital image data of the object.

In some embodiments, the method, further includes: accessing 2D digital image data of the object; and performing a 3D rendering technique to transform the 2D digital image data into 3D digital image data of the object; and wherein the fusion technique comprises affixing the 3D digital image data of the object to the view of the object in real-time.

In some embodiments, of the method, the fusion technique comprises matching a size of the view of the object in real-time with a size of the 3D digital image data, such that the size of the 3D digital image data is displayed in correct proportion with the size of the object.

In some embodiments of the method, the fusion technique comprises matching a shape of the view of the object in real-time with a shape of the 3D digital image data, such that the shape of the 3D digital image data is displayed in correct proportion with the shape of the object.

In some embodiments, the method further includes accessing a fixed reference marker near the view of the object in real-time, wherein the fixed reference marker provides sufficient data to provide a unique 3 dimensional orientation, and depth, of the view of the object, even as the position or orientation of the view of the object changes.

In some embodiments of the method, performing the fusing technique comprises utilizing the fixed reference marker to affix the digital image data to the view of the object in real-time.

### BRIEF DESCRIPTION OF THE DRAWINGS

The accompanying drawings are not intended to be drawn to scale. Like reference numbers and designations in the various drawings indicate like elements. For purposes of clarity, not every component may be labeled in every drawing. In the drawings:

FIG. 1 is an example of prior art machines that can be very useful during a surgical procedure, but are extremely cumbersome to use.

FIG. 2 is a high level block diagram of a system for aiding in surgical navigation, in some cases using AR elements, and in some cases facilitating remote viewing of the surgical site through VR, according to some embodiments.

FIG. 3 is a schematic illustration of an example surgical navigation system, according to some embodiments.

FIG. 4 shows an example block diagram of how the navigation system provides functionality to a remote location, according to some embodiments.

FIG. 5 is a photographic image of an example surgery room that utilizes the surgical navigation system, according to some embodiments.

FIG. 6 is an illustration of an example surgical platform where surgery is performed while using an AR screen that is part of the surgical navigation system, according to various embodiments.

FIG. 7 is an illustration of a closer view of the AR screen of FIG. 6, according to some embodiments.

FIG. 8 provides an example of how the screen may be transparent, or provide the appearance of transparency, while also enabling AR elements to be displayed.

FIG. 9 is a schematic diagram illustrating various modules of an all-in-one multifunctional apparatus, such as surgical navigation system apparatus or platform, according to various embodiments.

FIG. 10 is a schematic illustration of an example of a surgical navigation system apparatus or platform, according to various embodiments.

FIG. 11 is a schematic illustration of an example of a surgical navigation system apparatus or platform with additional features, according to various embodiments.

FIG. 12 is another schematic illustration of an example of a surgical navigation system apparatus or platform with additional features, according to various embodiments.

FIG. 13 is a schematic illustration of an example of a surgical navigation system apparatus or platform, with an example use case shown, according to various embodiments.

FIG. 14 shows an example scenario of a specialist or non-specialist wearing the headset navigation system, according to some embodiments.

FIG. 15 shows an example application of the navigation system, according to some embodiments.

FIG. 16 shows a block diagram of the surgical navigation system software at a high level, according to some embodiments.

FIG. 17 illustrates the registration module of the surgical navigation system software, which is a hybrid approach to the registration process, in accordance with various embodiments.

FIG. 18 illustrates an example data flow and working of the surgical navigation system software to deliver augmented reality navigation based on the rigid body/fixed markers in the scene and how the system is capable of communicating with multiple holographic devices simultaneously, in accordance with various embodiments.

FIG. 19 illustrates the data flow and working of how holographic projection is superimposed on to the real scene, using combination algorithms.

FIG. 20 shows a set of examples of advanced visualization functions that are enabled in the holographic mode, in accordance with various embodiments.

FIG. 21 illustrates the data flow and working of how the instrument (with markers) is used for navigation, in accordance with various embodiments.

FIG. 22 provides an example illustration of what a user is able to see using the navigation system of the present disclosure, according to some embodiments.

FIG. 23 shows examples of various degrees of opacity of one of the sets of image data superimposed on the skull that is regularly in view, according to some embodiments.

FIG. 24 provides another example of the navigation system providing multiple overlays, according to some embodiments.

FIG. 25 shows a device with four markets arranged non-symmetrically, which can be placed in a constant position near the target patient.

FIG. 26 shows an instrument that may be attached to the patient or onto a fixed position of the operating table also having four points as fixed visual cues.

### DETAILED DESCRIPTION

It is to be understood that the following disclosure provides many different embodiments, or examples, for implementing different features of the disclosure. Specific embodiments or examples of components and arrangements are described below to simplify the present disclosure. These are, of course, merely examples and are not intended to be limiting. For example, dimensions of elements are not limited to the disclosed range or values, but may depend upon process conditions and/or desired properties of the device. Moreover, the formation of a first feature over or on a second feature in the description that follows may include embodiments in which the first and second features are formed in direct contact, and may also include embodiments in which additional features may be formed interposing the first and second features, such that the first and second features may not be in direct contact. Various features may be arbitrarily drawn in different scales for simplicity and clarity.

Further, spatially relative terms, such as “beneath,” “below,” “lower,” “above,” “upper” and the like, may be used herein for ease of description to describe one element or feature's relationship to another element(s) or feature(s) as illustrated in the figures. The spatially relative terms are intended to encompass different orientations of the device in use or operation in addition to the orientation depicted in the figures. The device may be otherwise oriented (rotated 90 degrees or at other orientations) and the spatially relative descriptors used herein may likewise be interpreted accordingly. In addition, the term “made of” may mean either “comprising” or “consisting of.”

Disclosed is an overall hardware and software system for aiding in surgical navigation. The system may be configured to facilitate an AR/VR rendering of a surgical procedure at a remote location. Included in the system are one or more hardware components, where in some embodiments it is manifested in a wearable device such as a headset. In other embodiments it is manifested in a bar attachment to a mobile computer, such as a smart pad or a laptop. In some embodiments, the hardware includes a portable surgical navigation tool that can move easily from one surgical room to another. In addition, the system includes software configured to convert or fuse input data received by the hardware and supply imaging data for an AR or VR environment at a remote location. The various components of the system will be described in more detail, below.

System Overview

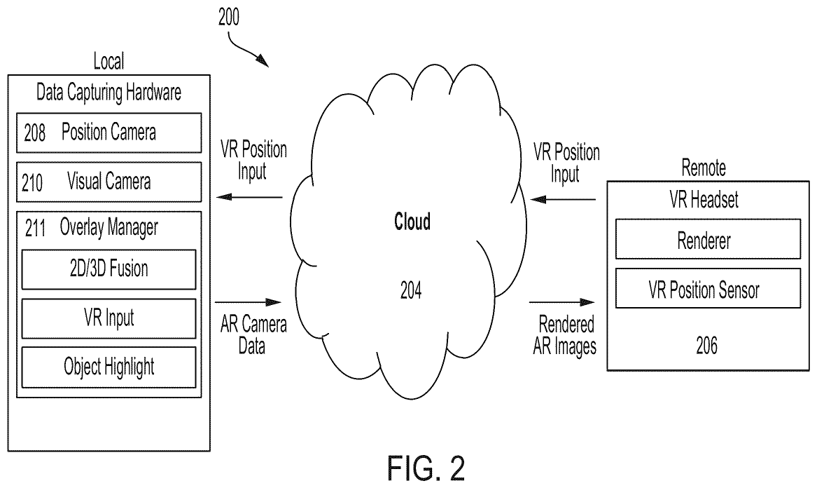

Referring to FIG. 2, shown is a high level block diagram 200 of a system for aiding in surgical navigation, in some cases using AR elements, and in some cases facilitating remote viewing of the surgical site through VR, according to some embodiments. On the local side (e.g., the location where the operation is being performed), aspects of the present disclosure include data capturing hardware, such as a headset having a position camera (e.g., a depth camera) 208 that collects position information and a visual or IR camera 210. Using the gathered position and visual information, an overlay manager 211 may process and render the images locally and overlay the images on the operation. In other cases, the data capturing hardware may include an attachment to a mobile computer with multiple sensors, such as the position camera and the visual camera. In other cases, the data capturing hardware may include a deployable surgical navigation system.

The data capturing hardware 208, 210 and overlay manager 211 may upload the rendered images to the cloud 204. At a remote location, the rendered AR images may be transmitted to a remote VR headset 218. The remote VR headset 218 may render the transmitted AR images in a 3-dimensional (3D) virtual reality space. A remote specialist, such as a surgeon located remotely, may interact with the VR display space. The remote surgeon may indicate the extent and depth of an incision on the VR images. The indicated position input provided by the remote surgeon may be transmitted to the cloud 204 and relayed to the local non-specialist, such as a medical student or technician operating the local data capturing hardware. The local overlay manager may then add the VR position input to the rendered AR images so that the non-specialist may use the VR position input in the procedure or operation.

While one use of the navigation system of the present disclosure is in the context of medical procedures, in general, it should be understood that these devices and procedures may be utilized for any operation where a specialist may be remote from a local non-specialist or vice versa. In some embodiments, the operation may be any remote operation. For example, the operation may be a manufacturing operation where a local manufacturer may need a specialist's instructions to manufacture a device having a specific geometry. In some examples, the operation may be a demolition or excavation operation, with the local non-specialist receiving instructions on where and how to place explosive charges. In some examples, the operation may be any other specialized operation that may benefit from accurate, precise, and real-time spatial or other instructions transmitted to an AR receiver.

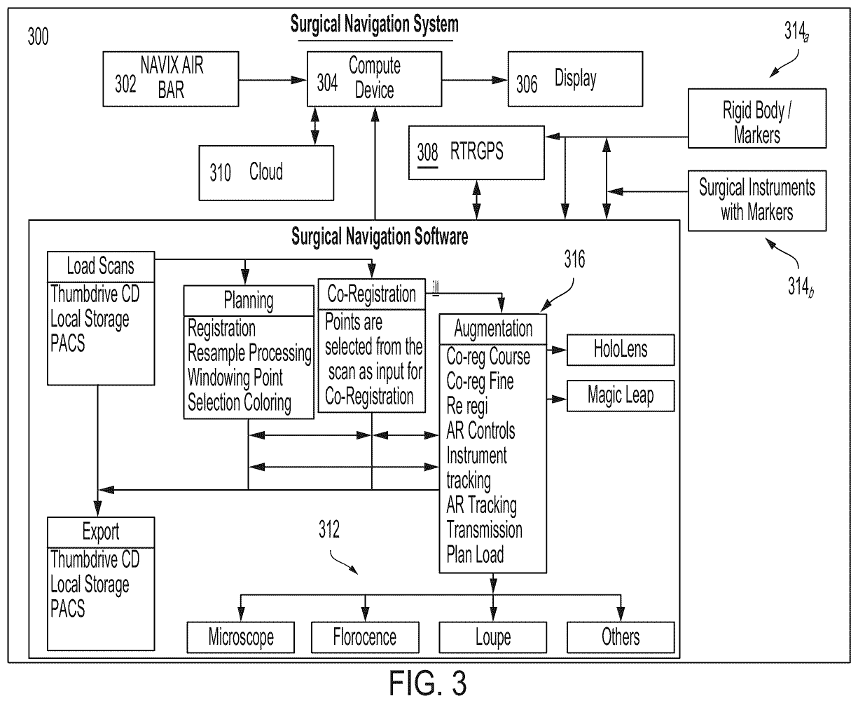

FIG. 3 is a schematic illustration of an example surgical navigation system 300. In accordance of various embodiments, the example surgical navigation system 300 can include a surgical navigation system apparatus or platform 302, a compute device 304, a display unit 306, a real time remote guided precision surgery (RTRGPS) 308, and/or cloud computing network 310.

The surgical navigation system 300 includes a multifunctional portable device 312 that delivers surgical navigation, magnification, fluorescence visualization and other functions, all in one device.

In some embodiments, the surgical navigation system 300 can weigh, for example, equal to or less than 130 lbs, though other sizes or weights can be contemplated based on each individual situation. The product 300 can be in the form of a small cart that can be transported if required to other areas of a hospital very easily. In other cases, the product can be in the form of an attachment to a mobile computer, such as a bar attachment. In other cases, the product can be in the form of a headset that a user can wear during a surgical procedure.

Below are some of the functions that can be accomplished with the surgical navigation system apparatus or platform, in accordance with various embodiments.

The device 300 is capable of doing surgical navigation with the help of markers 314 or using face detection, in accordance with various embodiments.

The device 300 is capable of doing magnification of surgical target area by up to 20× with optical zoom lens, in accordance with various embodiments.

The device 300 is capable of doing fluorescence visualization, in accordance of various embodiments.

The device 300 can be fitted with advanced functionalities such as, for example, confocal microscopy and Raman spectroscopy.

Multifunctionality allows the surgeon (user) conveniently and without any physical stress of complex positions to carry out the surgical procedure.

Augmented reality-based overlay 316 allows the surgeon to see the patient and perform surgery, thus reducing the time for surgeries increasing patient outcomes.

The device 300 can have a transparent display that will be used for augmented reality overlays in the surgical field of view, in accordance with various embodiments.

The device 300 also can use artificial intelligence-based segmentation of the organ anatomy and use that in surgical navigation to increase efficiency of the procedure, in accordance with various embodiments.

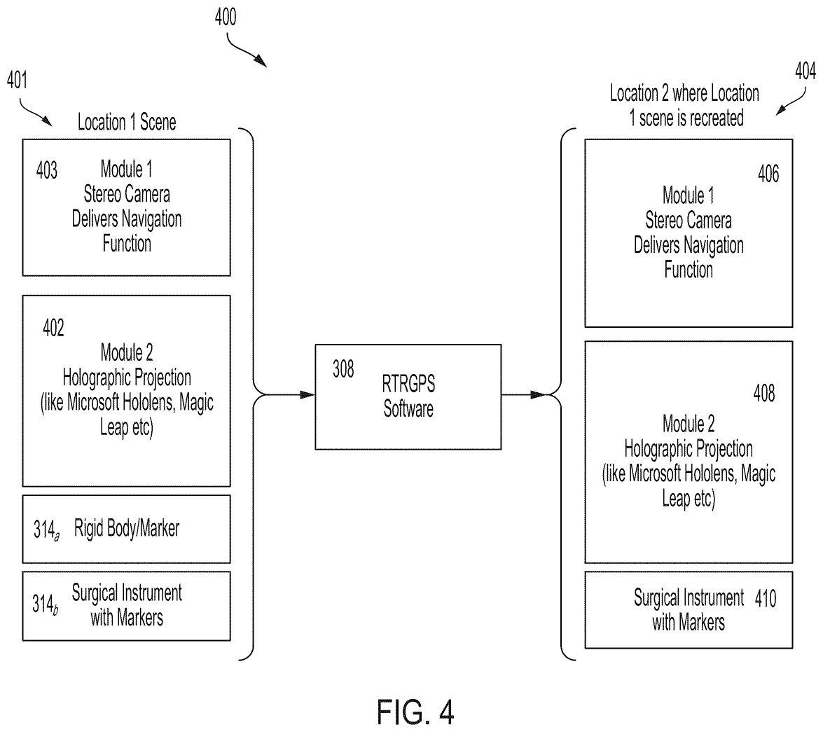

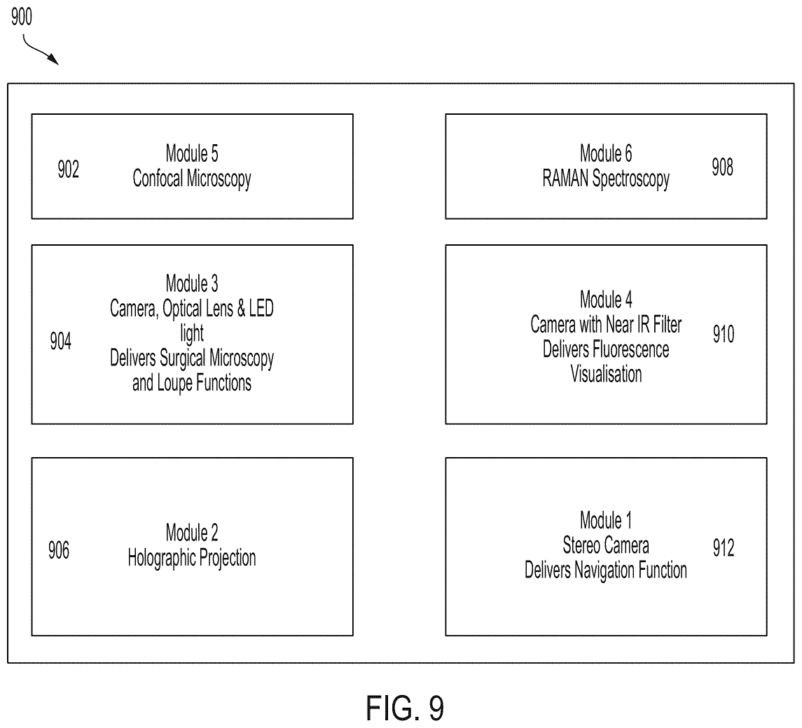

FIG. 4 shows an example block diagram 400 of how the navigation system 300 (FIG. 3) provides functionality to a remote location, according to some embodiments. FIG. 4 includes examples of various modules 402, 403 that represent distinct groups of functionality that may be available in certain versions of hardware and software of the present disclosure. A more comprehensive description of the kinds of modules available are described below, with respect to FIG. 9.

Here, the navigation device 300 (FIG. 3) is connected to the cloud or the PACS system, in accordance of various embodiments.

The user loads the scans using any of the common file storage systems like thumb drives or CDs or even cloud or PACS system, in accordance of various embodiments.

Once the scans are loaded, the user can either choose either to start planning or start Co-Registration or export to other forms so that they can continue on other surgical navigation systems, in accordance of various embodiments.

The user can start planning by selecting the planning option and using all the tools like point selection, windowing, coloring image processing and AI to plan the procedure that the user is planning on doing, in accordance of various embodiments.

The user can also share it with his/her peers or experts to get it approved, in accordance of various embodiments.

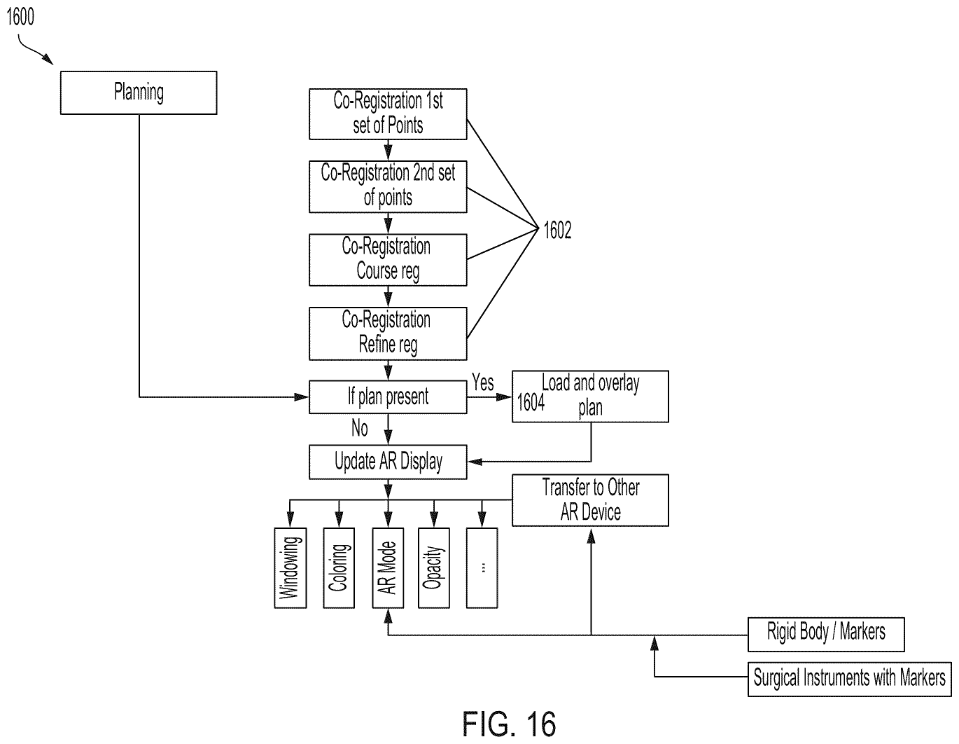

When the user wants to start the AR module 316 (FIG. 3) for the first time, the user can go through the Co-Registration module so that the initial set of points are selected and can start the AR module and overlay the volume, in accordance of various embodiments (see FIG. 16 and related description).

Once the AR module has been started, the user can switch between all the modules like planning, co-registration or augmentation 316 (FIG. 3).

In AR mode, the user can use the options provided to register the volume onto the patient with high degree of accuracy of 0.1 mm, in accordance of various embodiments.

Once all the setup has been done, the user can either continue using the system 300 (FIG. 3) or connect to any of the AR devices 402, like HoloLens or Magic Leap, to continue the procedure, in accordance of various embodiments.

The system can also be connected to the RTRGPS system 308 so that the user at location 2 can get an exact copy of the location 1 400, in accordance of various embodiments.

This connection with the RTRGPS system 308 can be used to sync any part of the application, in accordance of various embodiments.

As shown in FIG. 4, the RTRGPS 308 software module can take the data from location scene 1 400 and transfers this data over edge computing protocols (MQTT), for example, to recreate the location scene with depth perception at location 2 402. Further description of the software component of the present disclosure, that includes the RTRGPS 308 functionality, is described more below.

Location 1 400 can have either a surgical navigation system 300 or any other system that has the following modules/components at a minimum:
a. Module 1 403: Stereo Camera;
b. Module 2 402: Holographic projection;
c. Rigid Body/Marker 318ₐ;
d. Surgical Instruments with Markers 318_{b}.

Location 2 404 can either have a surgical navigation system of any other system that has the following modules/components at a minimum:
a. Module 1 406: Stereo Camera;
b. Module 2 408: Holographic projection;
c. Surgical Instrument with Markers 410.

Data from Location 1 is transferred over edge computing protocol (MQTT) via the RTRGPS Software.

Data must include at a minimum but not limited to:
a. Location 1 401 system orientation, translation information captured by Module 1 403. This is retrieved by the RTRGPS 308 Software when Module 1 identifies the Rigid Body/Marker.
b. Location 1 401 video stream as seen by Module 1 406.
c. Location 1 401: The orientation, translation information captured by Module 2 402, when it identifies the Rigid body/Marker 314a.
d. The orientation, transformation information captured by either Module 1 403 or Module 2 402 when the surgical instrument with markers 314_{b} enters the Location 1 401 scene.
e. Location 1 401 scene is the area that the user is going to perform the task.

This data is then transferred over edge computing communication protocols (MQTT) to Location 2 404 via the RTRGPS 308 Software.

At Location 2 404, the RTRGPS 308 software loads this data into the Module 1 406 and Module 2 408 to recreate the scene from location 1 401 with full depth perception using Module 2 408 holographic projection combined with a real live feed providing real true depth perception for user at Location 2 404.

Any surgical planning software or surgical navigation system 300 (FIG. 3) software provides all the data that is relevant to the surgical plan. A surgical plan includes but is not limited to Patient Scans and trajectory details.

Continuing with this scenario, now the 2 locations 401, 404 are synced. The sync has 0 latency on 5G speeds and the entire system can have more than 60 fps render speeds at 5G speeds.

In some scenarios the user at location 1 401 is guiding the user at location 2, for example, in a simulation.

In some scenarios the user at location 2 404 is guiding the user at location 1, for example, in a remote guidance situation with prevision.

At Location 1: The surgical instrument with markers 314_{b} is used by the user to perform the task at location 1 401.

Each marker/rigid body 314a may be a unique marker. Even the surgical instrument with a marker 314_{b} must be unique. No two markers 314 of the same type must be in a single location. The uniqueness may be derived from having four or points in combination, placed at unique distances in combination, from each other.

The RTRGPS 308 is continuously transmitting data and receiving data from both locations 401, 404 and syncing them at the same time.

In some scenarios the surgical instrument intersects a point P (p1, p2, p3) in space.

Space is the scene in location 1 401 or location 2 404. This point coordinates are accurately picked up by Module 1 403, 406 and Module 2 402, 408. The same point is virtually highlighted for guidance at the other location. The precision is as good as the precision of Module 2 402, 408 in identifying a point coordinate in space.

In some scenarios there can be more than 2 locations. There is no limit on the number of locations that can be connected through the RTRGPS 308 software.

Location 1 401 Markers: The markers or rigid body 314a, 314b must always be visible to the Module 1 404 and Module 2 402.

In some scenarios the unique features and contours of the scene in location 1 401 that do not change can also be used as rigid bodies/markers 314a, 314b.

In robotics systems where there are no visualizations available, the surgical navigation system 300 (FIG. 3) with markers 314a, 314_{b} can also be used to visualize the movements of the robotic arms inside the patient. This adds an extra 3D depth visualization to the robotic systems.

A team of trainees or medical students can practice in real time the surgical approach and nuances during surgical procedures under the guidance of the surgeon at location 1, or a surgeon at location 2 404 that is guiding the surgeon at location 1 401 during the surgery.

Location 1 401 and location 2 404 need not be pre segmented/labelled/marked with the RTRGPS 308 system. The system 300 (FIG. 3) enables real time depth scene rendering and precise guidance in both locations using holographic depth projections and Marker in 1 scene.

The user can use this to collaboratively work on the planning or the surgery or can be used for teaching or guiding the surgery, in accordance of various embodiments disclosed herein.

As long as the fixed marker is present in the view of the system the AR tracking is possible, in accordance of various embodiments disclosed herein.

If any of the instruments are to be used, then the instrument markers can be used to track the instrument after tracking, in accordance of various embodiments disclosed herein.

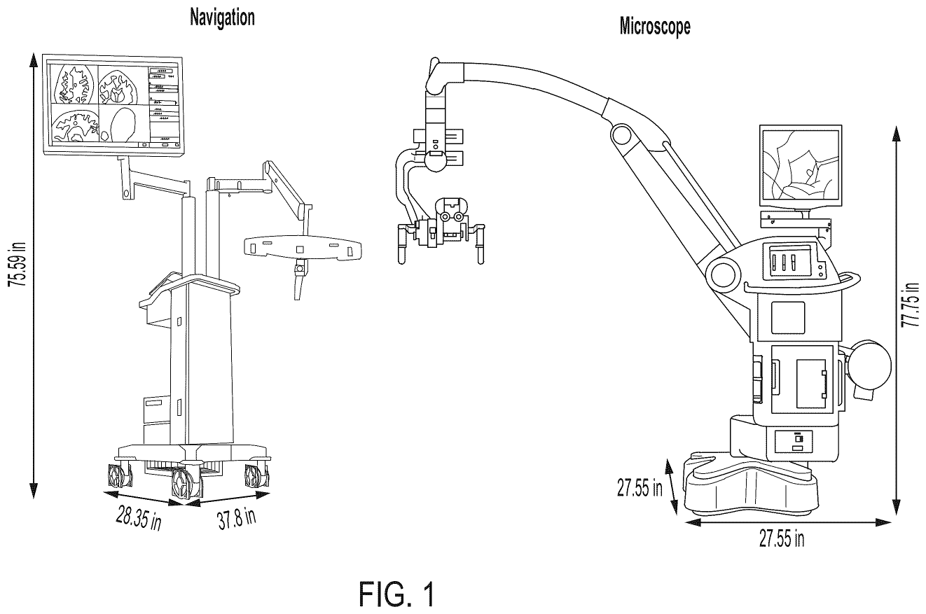

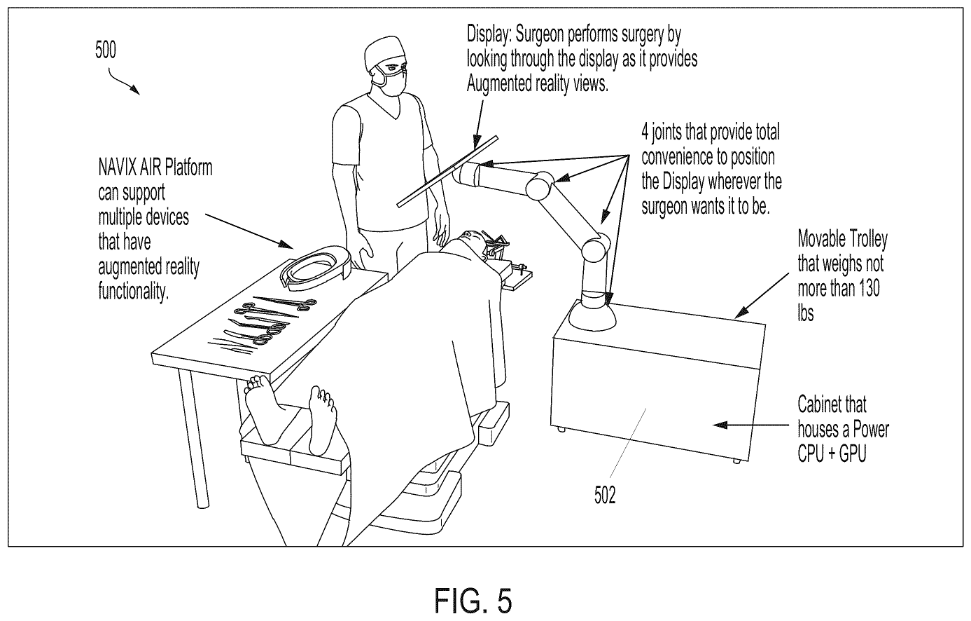

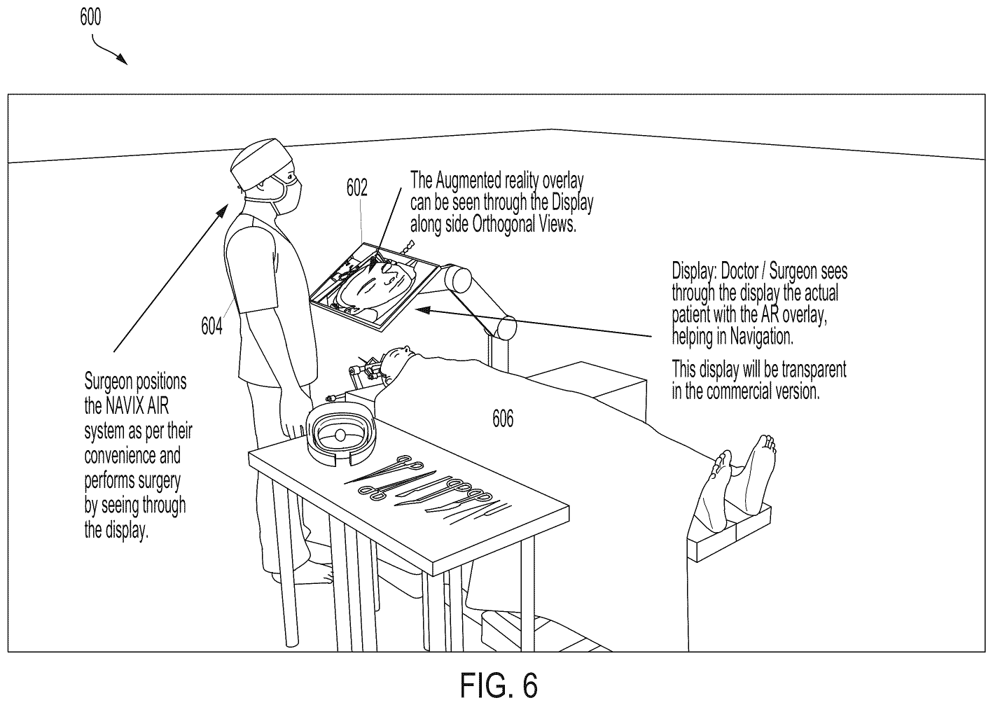

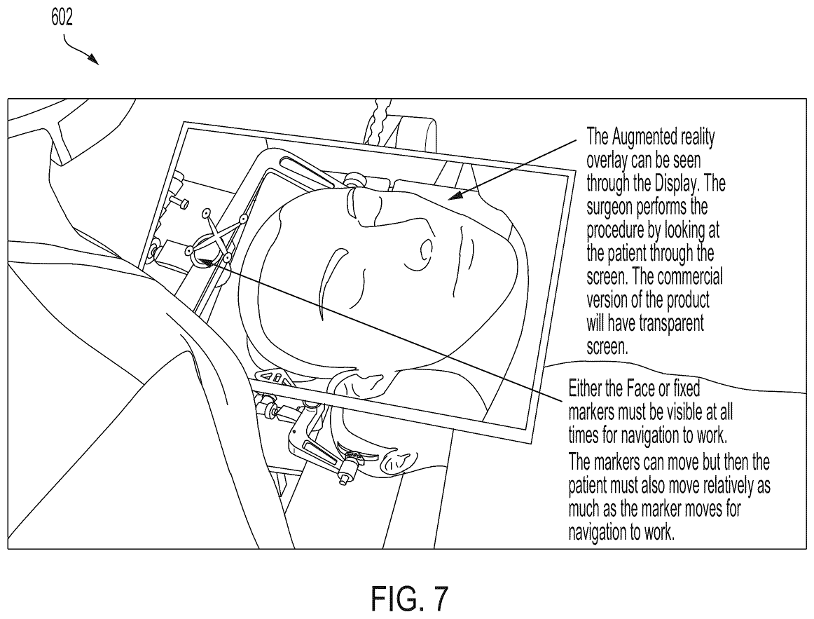

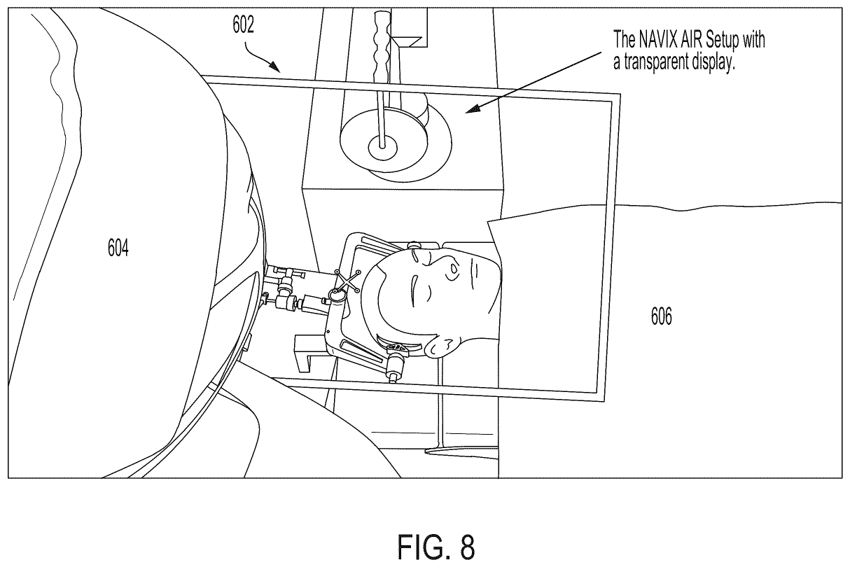

FIGS. 5, 6, 7, and 8 show various example scenarios of how the surgical navigation system of the present disclosure may be used in a surgical procedure context. FIG. 5 is a photographic image 500 of an example surgery room. The navigation system 300 (FIG. 3) hardware takes the form of a cart 502 that can be more easily deployable into different rooms, than compared to the conventional navigation and microscope machines (see FIG. 1). FIG. 6 is an illustration 600 of an example surgical platform where surgery is performed, according to various embodiments. Here, the hardware of the present disclosure includes a screen 602 interposed between the surgeon 604 and the patient 606. The screen may allow for AR elements to be added over the view of the patient 606. FIG. 7 is an illustration of a closer view of the AR screen 602, according to some embodiments. FIG. 8 provides an example of how the screen 602 may be transparent, or provide the appearance of transparency, while also enabling AR elements to be displayed.

More specific details of the example components of the navigation system 300 (FIG. 3) will now be provided. This description focuses on various hardware examples and software components that establish the overall system described herein.

General Hardware Description

In some embodiments, the hardware of the present disclosure includes a multifunctional portable device that delivers surgical navigation, magnification, fluorescence visualization and many more, all in one device.

The technology and methods disclosed herein relate to a multifunctional portable all-in-one device that can deliver multiple functions including, but not limited to, surgical navigation, surgical microscope, loupe, fluorescence visualization, pre op planning and/or simulations, as show for example in FIG. 9.

FIG. 9 is a schematic diagram 900 illustrating various modules 902, 904, 906, 908, 910, 912 of an all-in-one multifunctional apparatus, such as surgical navigation system apparatus or platform, according to various embodiments. As shown in FIG. 9, the surgical navigation system hardware apparatus or platform may include up to six modules 1-6 902, 904, 906, 908, 910, 912. In various embodiments, module 1 912 can include a stereo camera that is configured to deliver navigation functionality. In various embodiments, module 2 906 can include a holographic projection system, such as but not limited to, Microsoft Hololens, Magic Leap, etc. In various embodiments, module 3 904 can include a camera, optical lens, and/or LED light and is configured to function as a surgical microscope and/or to provide Loupe functions, e.g., magnifying to see small details. In various embodiments, module 4 910 can include a camera with an infrared (IR) filter and is configured for fluorescence visualization.

In various embodiments, module 5 902 can be configured for a confocal microscope or can be configured for confocal microscopy. In various embodiments, module 6 908 can include a Raman spectroscope or is configured for Raman spectroscopy.

Bar Attachment Hardware

In various embodiments, the modules 902, 904, 906, 908, 910, 912 of the surgical navigation system 300 (FIG. 3) apparatus or platform, as shown in FIG. 9, can be combined to fit into a minimalist horizontal bar form factor that can help achieve various advanced functionalities, such as those discussed above, within a single device. In various embodiments, the various modules 902, 904, 906, 908, 910, 912 of the surgical navigation system 300 (FIG. 3) apparatus or platform can be powered from a single laptop/desktop/tablet/high performance system. In various embodiments, the surgical navigation system 300 (FIG. 3) apparatus or platform can be fully customizable to include all the hardware modules 902, 904, 906, 908, 910, 912. In various embodiments, the surgical navigation system 300 (FIG. 3) apparatus or platform can include just some of the hardware modules 902, 904, 906, 908, 910, 912, depending on the user requirements. The surgical navigation system 300 apparatus or platform in the form of the bar attachment is ergonomic and very aesthetic in design because of its cuboidal shape and can be latched/attached to a display 602 (FIGS. 6-8) or tablet/laptop to work. The unique design of the surgical navigation system 300 (FIG. 3) apparatus or platform allows surgeons to operate without any restrictions in the surgical field of view, allowing for free movement of instruments in the surgical field of view.

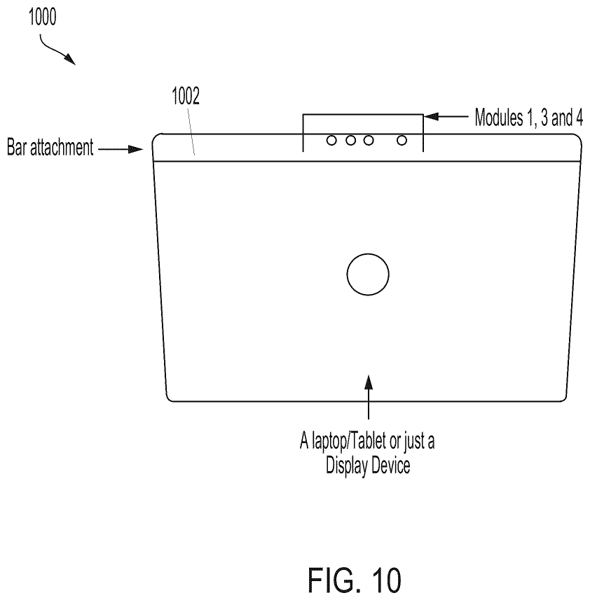

FIG. 10 is a schematic illustration of an example of a surgical navigation system apparatus or platform 1000, according to various embodiments. As shown in FIG. 10, the bar attachment 1002 may connect to the top of a laptop or tablet. the surgical navigation system apparatus or platform 1000 in this bar attachment 1002 form factor includes modules 1, 3, and 4 912, 904, 910. In various embodiments, the surgical navigation system apparatus or platform 1000 is attached to a display 602 (FIGS. 6-8) or laptop or tablet to any side, but ergonomically the top of the display 602 (FIGS. 6-8) or laptop or tablet may be a more intuitive location to attach or latch.

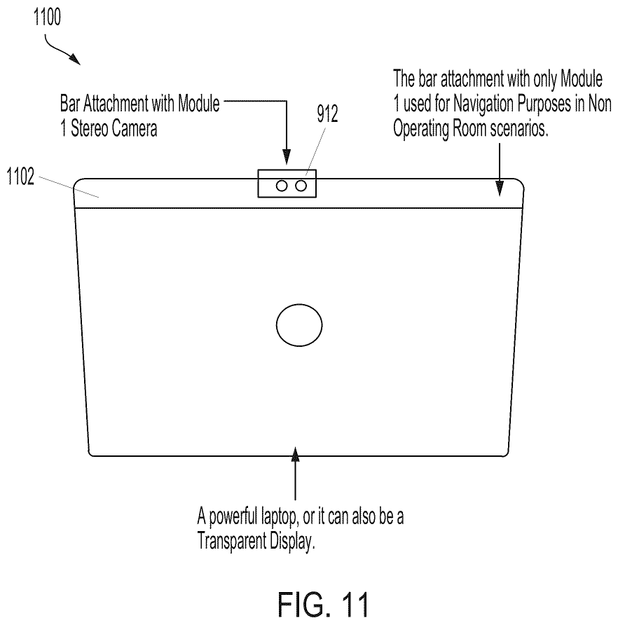

FIG. 11 is a schematic illustration of an example of a surgical navigation system apparatus or platform 1100, according to various embodiments. As shown in FIG. 11, the surgical navigation system apparatus or platform 1100 in the form of the bar attachment 1102 in this example includes module 1 912, e.g., a stereo camera, attached to, for example but not limited to, a laptop, tablet or a display device.

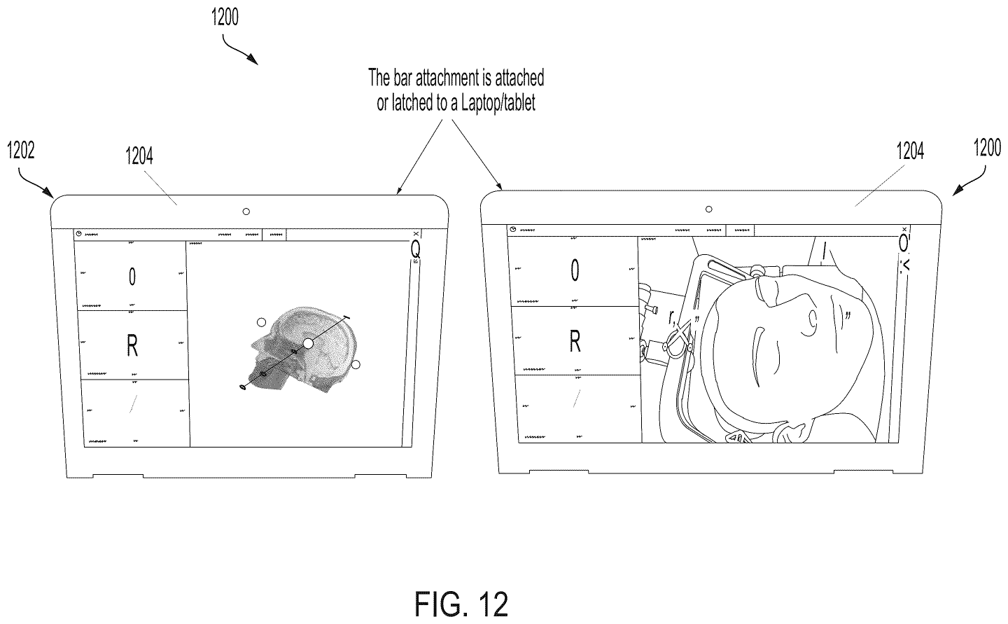

FIG. 12 is a schematic illustration of an example of a surgical navigation system apparatus or platform 1200, according to various embodiments. As shown in FIG. 12, the navigation system 1200 can include a laptop 1202 showing various views of an operation. As illustrated in FIG. 12, the bar attachment portion 1204 may be attached or latched to, for example but not limited to, a laptop or a tablet 1202.

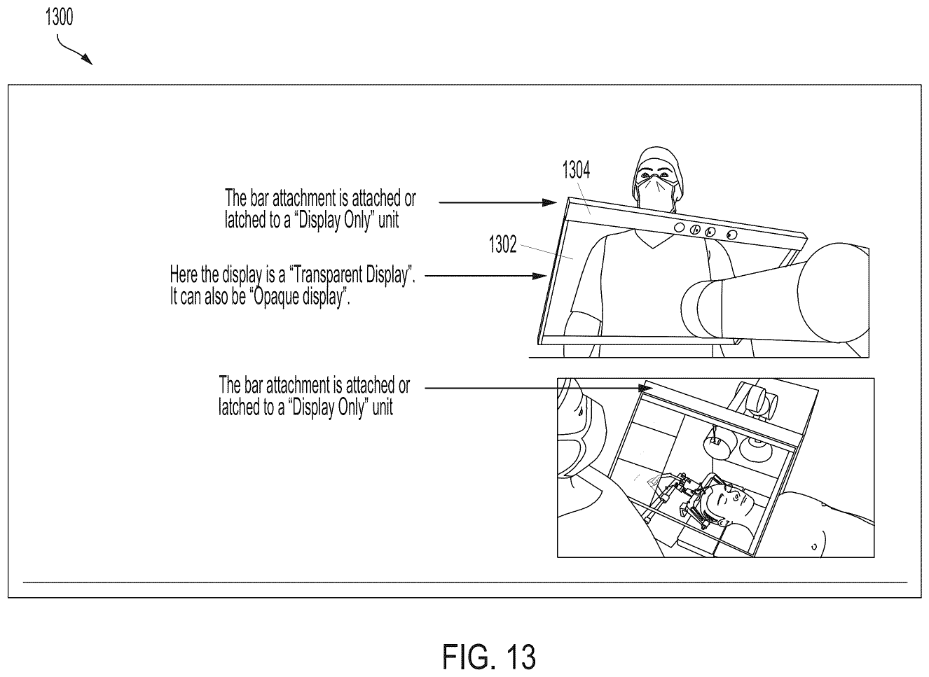

FIG. 13 is a schematic illustration of an example of a surgical navigation system apparatus or platform 1300, according to various embodiments. As shown in FIG. 13, surgical navigation system apparatus or platform 1300 in the form of the bar attachment 1304 can include a display unit 1302, e.g., a transparent display or an opaque display, showing various views of an operation. As illustrated in FIG. 13, the surgical navigation system apparatus or platform 1300 can be attached or latched to the display unit 1302.

In various embodiments, the surgical navigation system apparatus or platform 1300 can be configured to connect the various hardware modules through USB or other communication ports to a computing device 304 (FIG. 3), such as those shown in FIGS. 10, 11, and 12. As stated above, the computing device 304 (FIG. 3) can be, for example but limited to, a laptop, tablet, desktop or high performance computer system. Alternatively, the bar attachment can also be attached onto a display 1302 only system, as shown in FIG. 13. In various embodiments, the display and the surgical navigation system apparatus or platform 1300 are connected to a high performance computer system.

Headset Hardware

In some embodiments, the surgical navigation system apparatus or platform may be manifested in a headset that may be worn in the operating room. To help facilitate remote instruction of a local non-specialist by a remote specialist, the headset navigation system according to some embodiments may be configured to collect spatial and visual or near IR data. To collect the data, one or more cameras may be attached to the headset. The headset may be configured to display AR elements in the field of view. The cameras may be oriented to collect position and visual or near IR data in the direction that the remote non-specialist is facing.

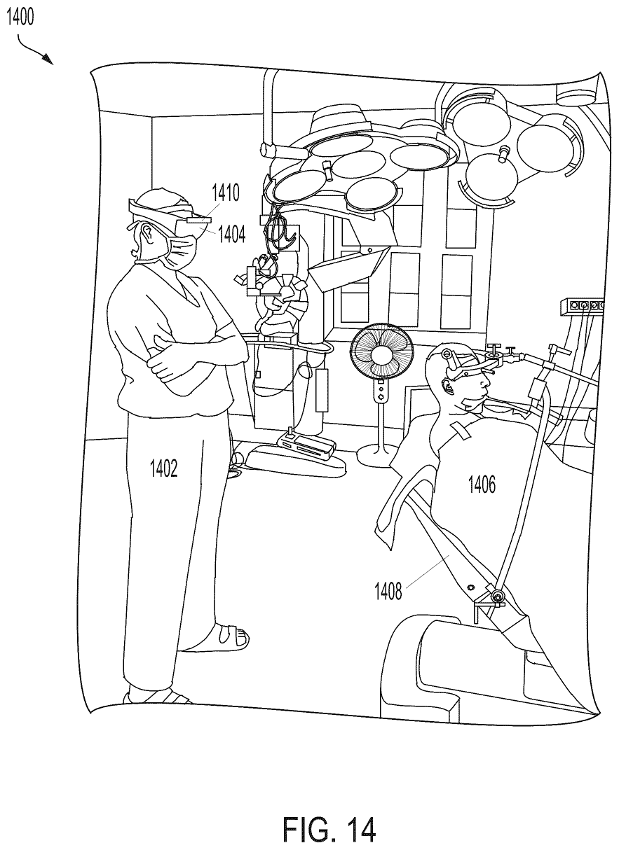

FIG. 14 shows an example scenario 1400 of a specialist or non-specialist 1402 wearing the headset navigation system 1404, according to some embodiments. The headset wearer 1402 is able to see the patient 1406 on the operating table 1408 while also seeing AR elements in the field of view, as displayed through the headset 1404. In some embodiments, the image data captured by the headset may reflect what the user sees, based on the orientation of the camera sensors 1410. These image data may be transmitted to a remote location, through the cloud for example, and used to display a VR rendition of what is being seen in the OR, to the other user at the remote location.

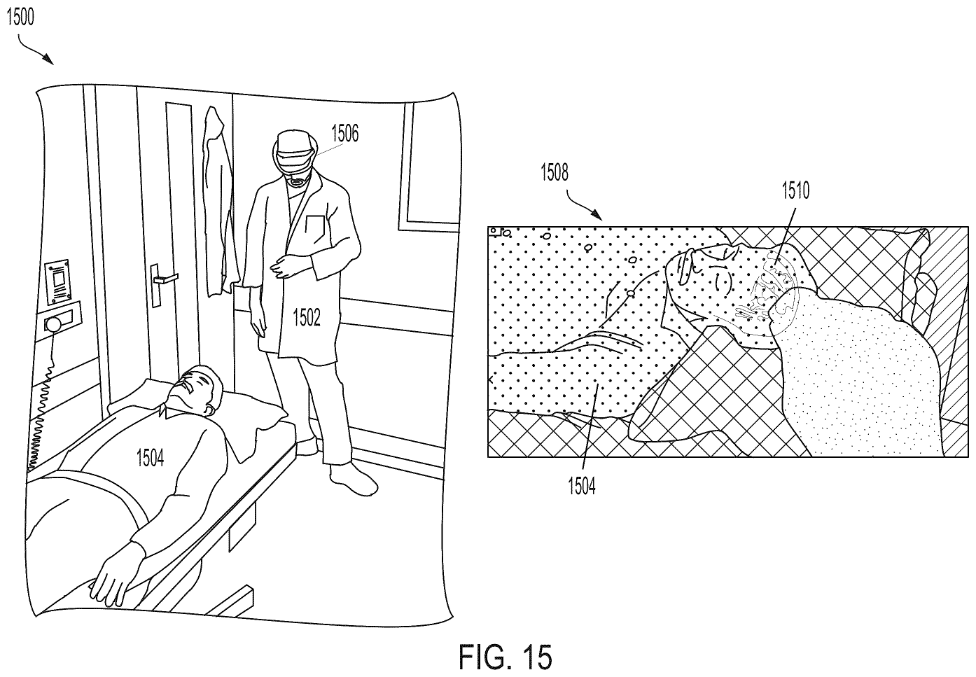

FIG. 15 shows an example application 1500 of the navigation system, according to some embodiments. The example scenario on the left shows a specialist 1502 tending to a patient 1504 while wearing the navigation system in the form of the headset 1506. The specialist 1502 sees the patient 1504, but can also see other elements. Shown in the right is an example of the first person view 1508 of the specialist 1502 through the headset 1506, which also includes AR elements 1510. Here, an approximate position of the of patient's brain 1510 is overlaid onto the patient 1504, at a position where the brain 1510 has been measured to be, relative to other reference points of the patient 1504. The overlay of the patient's brain 1510 may be a 3D rendering, such that the specialist 1502 wearing the headset 1506 may walk around the patient 1504, and in real time the various angles of the brain 1510 will change according to the orientation of the headset 1506 relative to the patient 1504. Example implementations for achieving this overlay 1508 will be described further below.

In some embodiments, the image data of the patient 1504 and one or more scans of the patient 1504 in other forms, such as an x-ray or an MRI, may all be transmitted to a remote location. A user at the remote location (e.g. location #2 404 in FIG. 4) may utilize the navigation system (e.g., system 300 of FIG. 3) according to the present disclosures, either in the form of the headset 1506 or the bar attachment (FIGS. 10-12), and see an overlay (e.g., overlay 1508) of the one or more scans on top of the patient 1504 in the precise placement relative to the patient 1504. This may allow the remote user to make better decisions about how to treat the patient 1504, even from a remote location.

The cameras attached to the AR headset 1506 may be any type of position and/or visual or near IR data sensing cameras. For example, an existing camera may be connected to the AR headset 1506. In some embodiments, the position camera may be any type of camera that may collect position and depth data. For example, the position camera may be a LIDAR sensor or any other type of position camera.

In some embodiments, the visual or near IR camera may be any type of visual camera. For example, the visual or near IR camera may be a standard visual camera, and one or more filters may be placed on the visual camera to collect near IR information. In some examples, a camera may be configured to specifically collect IR data.

In some embodiments, adding cameras to the AR headset 1506 may add additional weight to the AR headset 1506. Adding weight to the AR headset 1506 may decrease the user's comfort. For example, the additional weight may increase the user's neck fatigue. Furthermore, the additional weight may reduce the stability of the AR headset 1506 on the user's head, causing it to slip and reducing the quality of the collected data.

In some embodiments, a single camera or camera housing for each camera may be built into the headset 1506, used to collect position and visual or near IR data. The headset 1506 may include two cameras in the same housing that collect data through a single lens. This may reduce the weight of the AR headset 1506. Reducing the weight of the AR headset 1506 may help to improve the comfort of the user and reduce the slippage of the AR headset 1506 on the user's head.

In various embodiments, the surgical navigation system apparatus or platform (e.g., system 300 of FIG. 3), in the form of the bar attachment (FIGS. 10-12) or headset 1506, or other variant, can include module 1 (or only module 1, see FIG. 9) for extreme portability, e.g., for small interventions to be performed by a user in a non-operating room setting. This configuration provides the user, e.g., a surgeon, with navigation functionality. In accordance with various embodiments, the surgical navigation system apparatus or platform (e.g., system 300 of FIG. 3) is configured to perform only the navigation function.

In various cases of intervention, module 2 (see FIG. 9) can also be included in the surgical navigation system apparatus or platform (e.g., system 300 of FIG. 3) to provide holographic projection. In various embodiments, the user or the surgeon 1502 can use augmented reality overlay for navigation functions.

In cases, for example, where the user 1502 is in the operating room and requires most of the multiple functions to perform the surgery effectively, the surgical navigation system apparatus or platform (e.g., system 300 of FIG. 3) can therefore be configured to include all modules 1-6.

While components for all or some modules may be available using conventional products, manufactured for miniature form factor to enable portability, these components are combined into an intuitive form factor that enables these advanced functionalities to be achieved with one device. For example, the bar attachment (FIGS. 10-12) can be powered from a single laptop/desktop/tablet/high performance system. The bar (FIGS. 10-12) is ergonomic and very aesthetic in design because of its shape and can be latched/attached to an AR head mounted display to work. The placement of the modules in the described embodiments allows surgeons 1502 to operate without any restrictions in the surgical field of view, allowing for free movement of instruments in the surgical field of view.

Software for Image Collection and Rendering

As part of the surgical navigation system, and according to some embodiments, planning and processing software is disclosed and provides solutions for transforming the input data of the hardware, such as the received stereo camera data, into a more helpful visual display that overlays multiple sets of data together. In addition, the software described herein may enable the remote connection to local views in the operating room.

In some embodiments, the surgical navigation system software includes planning software. Prior to any procedure, a plan is required. This plan is generated or approved by the surgeon performing the procedure. Planning software often requires the patient's 3D Scans (e.g., magnetic resonance (MR) and computerized tomography (CT)) and/or 2D scans (e.g., X-ray and Ultrasound).

All MR and CT scans can be provided in the Digital Imaging and Communications in Medicine (DICOM) format as an example, which is an international accepted format.

The software in some instances can be available either on a local system (e.g., laptop, desktop, tablet) or on the cloud.

The software can connect to the PACS (Picture and Archive Communication System) that stores the medical images. The software can query the PACS system and download the patient 3D images.

The user now has options to view the 3D scans on the device (e.g., laptop, tablet, desktop) that may be a part of the navigation system. The user has access to standard image processing tools to manipulate the DICOM images such as, for example, windowing, zoom, pan, scroll, line, point selection.

The user can create trajectories by choosing target and entry points to review the trajectory with the team aiding in the procedure.

In addition, in some embodiments, the software can process real time imaging data of the patient in the operating room, and can combine the 3D and/or 2D images with the real time image data of the patient, and can accurately overlay where the 3D and 2D images should be shown within the proper locational context of the patient's body.

This plan can be saved in a HIPAA compliant database that can either be local on the device or can be saved on a HIPAA compliant cloud.

The plan can be exported to a removal storage media from a local device and can be used at other surgical navigation planning stations or can be directly accessed from the cloud on other surgical navigation planning stations. The plan saved in the database has all the data that is required to reload the plan as it was saved by the user thus saving time on repeating the same tasks inside the operating room.

The disclosed surgical navigation system software has some advanced functions for medical image processing that will help the user/surgeon in accurate and faster planning.

FIG. 16 shows a block diagram of the surgical navigation system (e.g., system 300 of FIG. 3) software at a high level, according to some embodiments. FIG. 16 shows how data in the software system flows between the different modules of the system, in accordance with various embodiments disclosed herein.

Referring to FIG. 16, in some embodiments, the software performs a registration process 1600 as part of its processing algorithm. Registration 1600 can be used to describe a process whereby two scans of the same patient are superimposed to have the same coordinate system (or fusion) such that the features of the two scans are superimposed. There are multiple scans acquired because each scan might be different in the acquisition protocols used, with examples including T1 MRI, T2 MRI, DWI MRI, CT PLAIN, CT CONTRAST, FMRI, DTI MRI, etc. Co-registration 1602 may refer to coordinating multiple sets of data to be coordinated at one, two, or three or more common points of reference relative to the patient. Combined with the plan of how to perform the surgical procedure 1604, the software may then place the various sets of co-registered data in the context of a surgical site on the patient. The software may then direct processing to mainly this area, so that in the AR display 1506 (FIG. 15) available to the surgeon or other user of the navigation system hardware, the user may then be able to see through the AR display the various co-registered data sets that are relevant to the surgical site. Rigid body markers, and/or rigid surgical instrument markers, may be used to objectively orient the various sets of data during the co-registration process 1602, and then may continue to be relied on when performing the real-time AR displays.

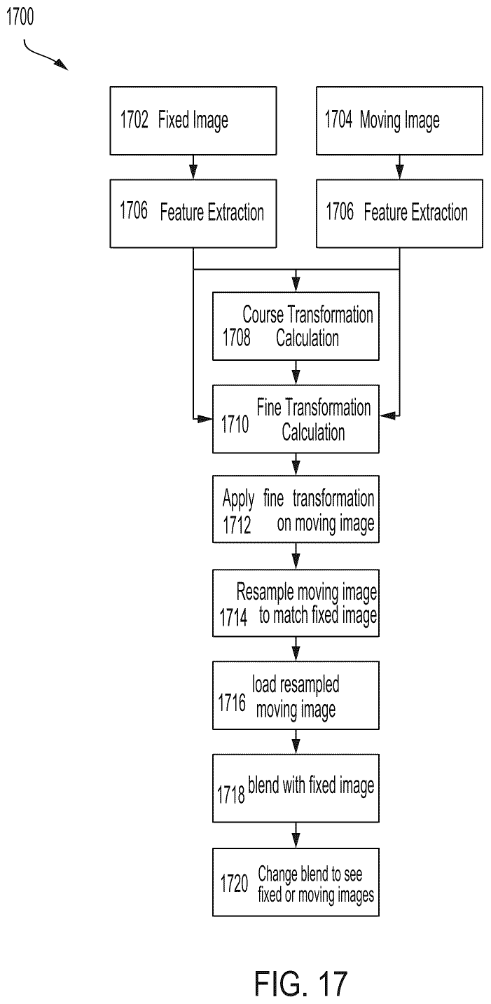

FIG. 17 illustrates the registration module of the surgical navigation system software, which is a hybrid approach to the registration process 1700, in accordance with various embodiments. Here, the software may access a fixed image 1702 from recorded 2D or 3D images, and combine them with a moving image 1704, such as real-time data being viewed through the navigation system hardware (e.g., headset 1506 of FIG. 15). In software terminology, if there are two patient scans that are to be fused, one is typically referred to as a fixed scan and the other scan is a moving scan. The moving scan typically is the scan to which the algorithm derived rotation and translation (or together referred to as transformation) is applied so that the moving scan can fuse with the fixed scan.

Feature extraction 1706 may be performed for both images to identify key features to pivot off of. Transformations 1708, 1710 both high fidelity and low fidelity, may be performed to convert the images into a common set of data. The software 1700 may then apply a fine transformation 1712 on the moving image 1704 to better calibrate the image to a closest known fixed image. A resampling 1714 of the moving image 1704 may be performed to find a best match to a fixed image 1702. The resampled image may be loaded to be compared 1716 with the fixed image 1702, and then blended 1718 with the fixed image 1702. The blended image may be changed 1720 in terms of opacity of one over the other, as desired, according to some embodiments.

The algorithm used for the registration process 1700 can be, for example, a custom hybrid algorithm used by the surgical navigation system. In a, for example, two-step process, the first step is a coarse registration method 1700 that allows the bringing of the two scans closer to the same coordinate system. But, in certain circumstances, the output of this method 1700 does not provide accurate results to move forward, as this step can run on a small set of features and only has to do coarse estimation, thus taking very less time.

The second step is a fine tune registration method 1710 that the fine tuning of the two scans to come as close as possible such that they share the same coordinate system and the features are superimposed. This step can run with a large set of features that have to be matched between the two scans.

A typical registration processes can take 3-4 minutes, however the registration process discussed herein, in accordance with various embodiments, reduces the time taken by up to 60% on an average compute.

Realignment: In some scenarios the scan is acquired in a said orientation and the user wants to realign the scan to another preferred orientation. In the 3D world, orientation changes the way the world is perceived. Even the most advanced users tend to get confused when they look at the same organ/scene from a different alignment. Realignment is done by using the concept of a plane. The 3D scan is realigned by using the reference plane provided by the user. Planes can be defined with minimum of three points.

Surgical navigation system (e.g., system 300 of FIG. 3) realignment can ask for two points from the user. The third point can be automatically selected by the software as the mid-point of the two points selected, with an increment of 0.1 mm in the z-axis. If Point 1 is referred by coordinates p1, p2, p3 and Point 2 is referred by coordinates a1, a2, a3, then the third point to form a plane can be chosen automatically by doing ((p1+a1)/2, (p2+a2)/2, (p3+a3)/2+0.1 mm). This approach leads to highly accurate plane.

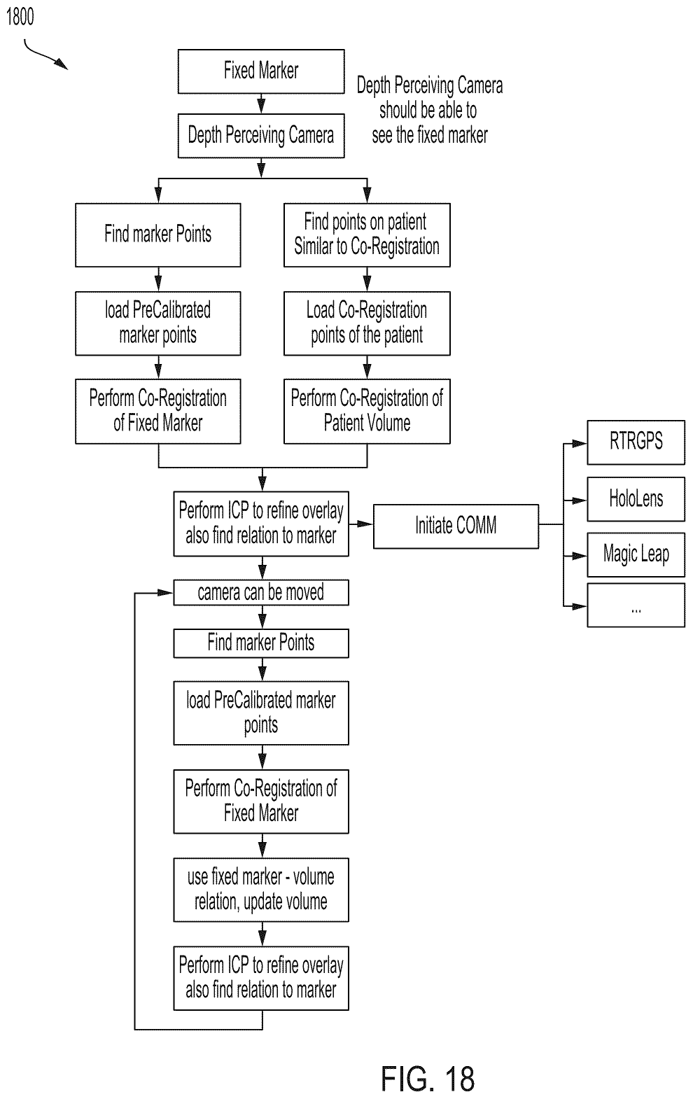

To effectively produce the augmented reality overlay, a co-registration can often be used such that the hologram is superimposed onto the real scene. FIG. 18 illustrates an example data flow and working of the surgical navigation system software to deliver augmented reality navigation based on the rigid body/fixed markers in the scene and how the system is capable of communicating with multiple holographic devices simultaneously, in accordance with various embodiments.

Co-registration (e.g., 1602 of FIG. 16) can take two sets of points as inputs, the first set of points including the point selected on the scan and the second set including the points in the real world which are selected with the help of the augmentation module.

After the points are selected, the system (e.g., system 300 of FIG. 3) can take two steps to overlay the 3D volume with high degree of accuracy of close to 0.1 mm.

In the first step, as the points are loosely selected, the system (e.g., system 300 of FIG. 3) can do a coarse estimation by using the two sets of points and gets the 3D volume as close as possible, in accordance with various embodiments.

In the second step, which can be referred to as the refinement step, the system (e.g., system 300 of FIG. 3) generates a 3D point cloud from the augmentation module and a 3D point cloud from the scans and uses this to refine the co-registration to get high degree of accuracy for overlay, in accordance with various embodiments.

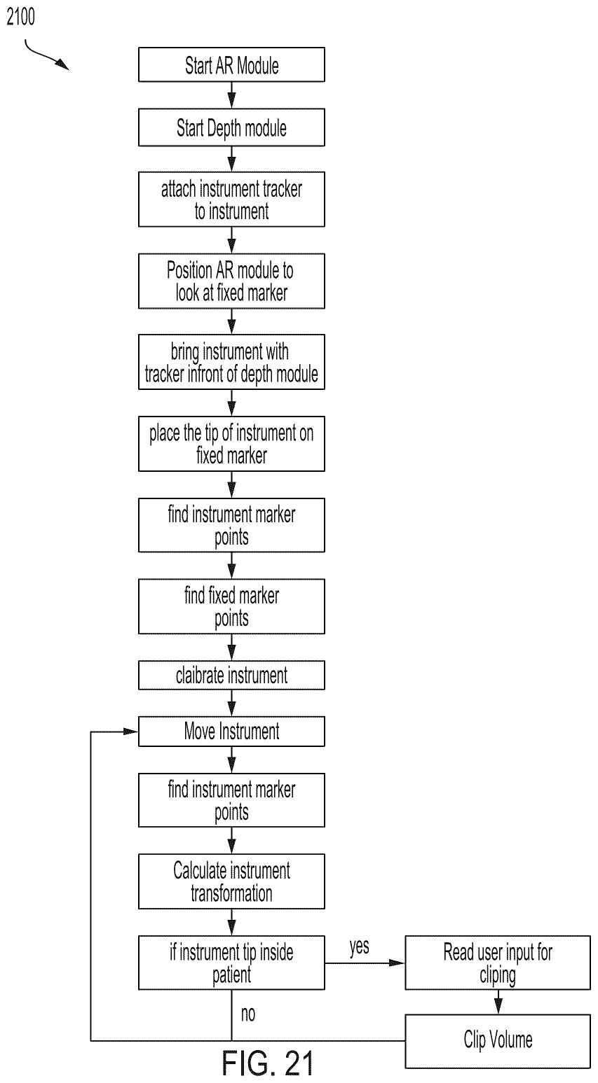

There are various options given for the user to control the augmented overlay. These options include, for example, opacity, clipping size, coloring, windowing, refine registration, AR Mode. FIG. 21 illustrates the data flow 2100 and working of how the instrument (with markers) is used for navigation, in accordance with various embodiments.

In holographic mode, the scans can be used to create a more detailed 3D volume that highlights different parts of the scans and colors them differently. This can help some users visualize different parts of the anatomy more clearly, in accordance with various embodiments.

Once the plan has been created and the 3D volume overlaid accurately, the system (e.g., system 300 of FIG. 3) can load the plan automatically and overlay it as well with the 3D volume, in accordance with various embodiments.

While this is being done, the fixed 3D marker will generally remain in view, and the system can use the relative orientation of the overlay with the fixed marker to make it a subsystem of the fixed marker, in accordance with various embodiments.

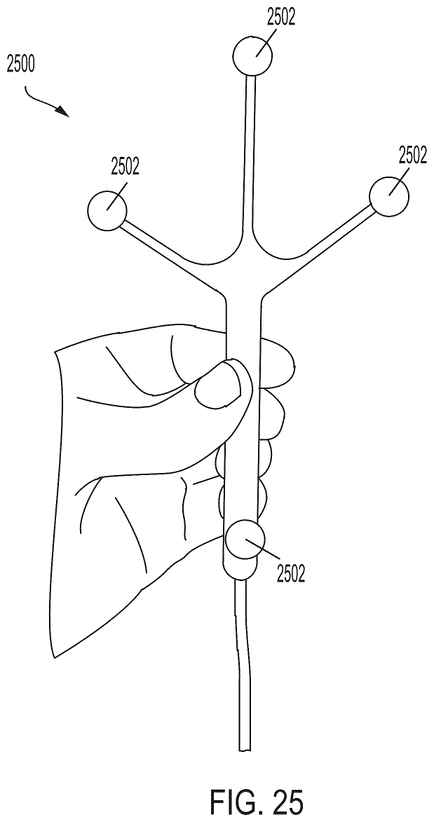

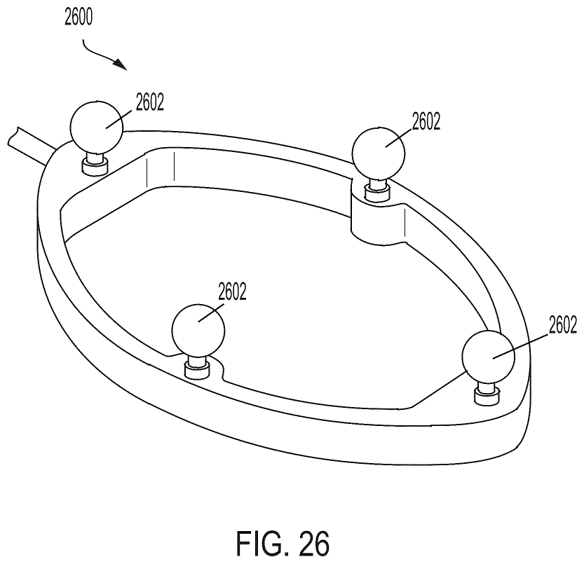

The user can then move around the fixed marker while the system updates the orientation of the holographic overlay with respect to the fixed marker, in accordance with various embodiments. Examples of a fixed marker are shown in FIGS. 25 and 26, and will be revisited below.

When the user has selected a good position to view and perform the procedure, the user can fix an instrument tracking marker to the instrument the user wants to use, in accordance with various embodiments. These fixed markers may be similar to ones shown in FIGS. 25 or 26 for example.

The system can track the instrument in real-time and can update the holographic overlay accordingly. See FIG. 21.

In such a way, the user can see the user's positioning inside the patient more clearly, in accordance with various embodiments.

If at any point in time the holographic overlay get misaligned, the user can trigger correction and the system quickly fixes the issue and get the accuracy back to near 0.1 mm.

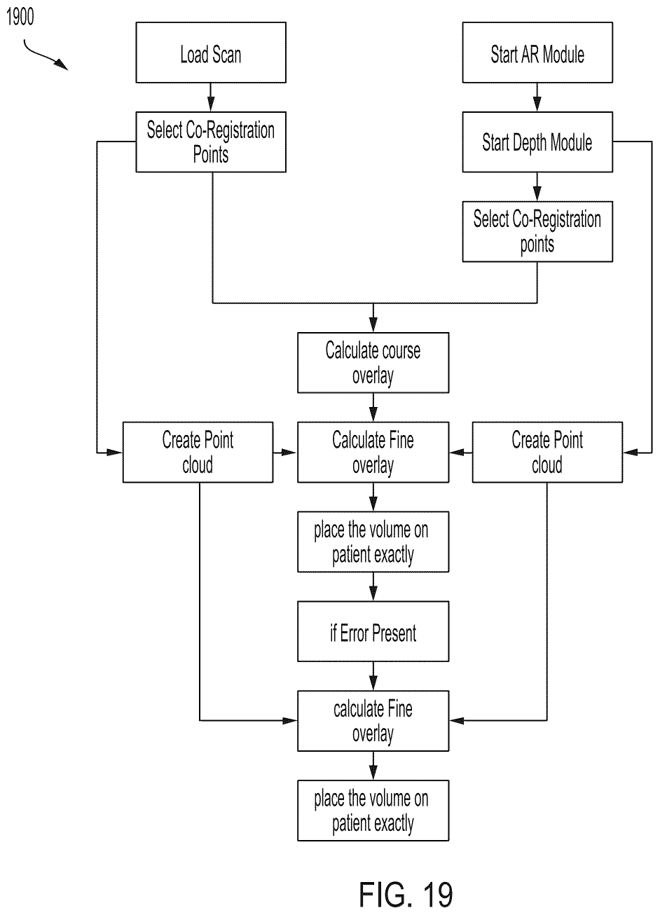

FIG. 19 illustrates the data flow 1900 and working of how holographic projection is superimposed on to the real scene, using combination algorithms. For example, CPD (Correlating point drift algorithm) and ICP (Iterative Closest Point algorithm), may be utilized, in accordance with various embodiments.

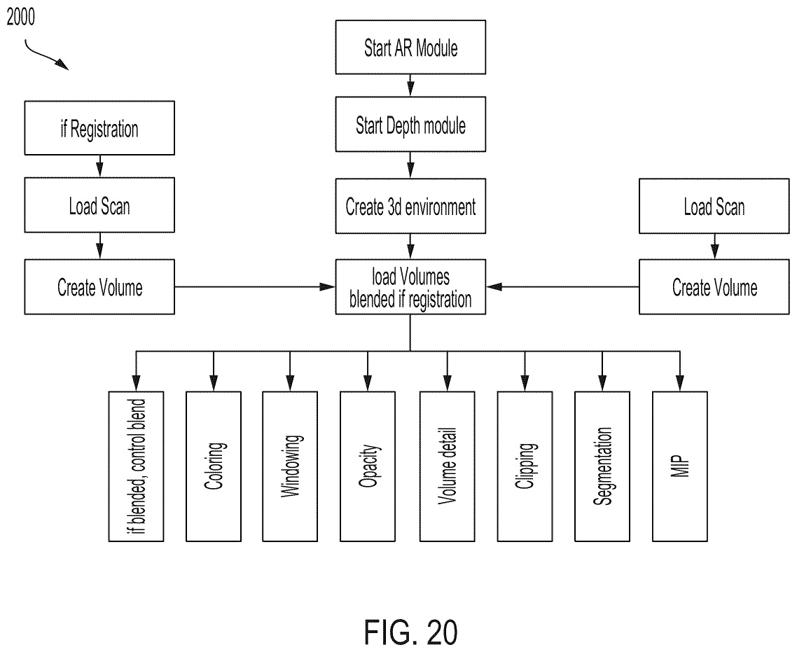

FIG. 20 shows a set of examples of advanced visualization functions 2000 that are enabled in the holographic mode, in accordance with various embodiments. The software of the present disclosure may also be configured to adjust settings in the AR environment according to these various settings.

The user can now connect any number of other AR devices like HoloLens or Magic Leap (see FIG. 18) and, using the fixed marker as reference, continue with the procedure with the AR overlays available as significant aides.

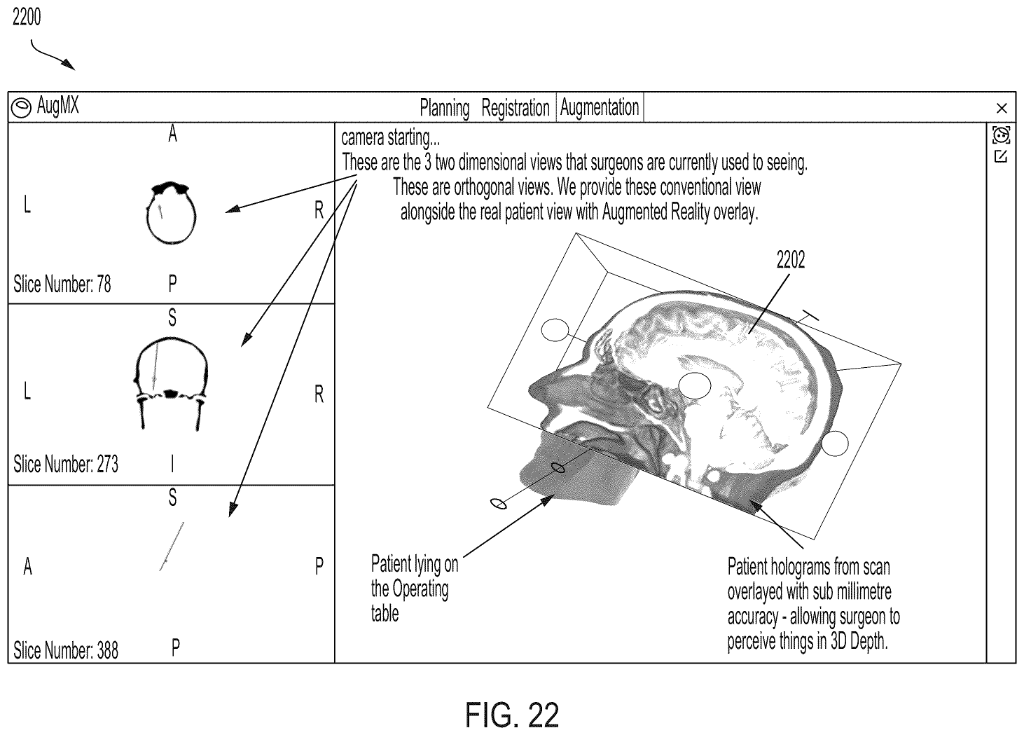

FIG. 22 provides an example illustration 2200 of what a user is able to see using the navigation system (e.g., system 300 of FIG. 3) of the present disclosure, according to some embodiments. Shown here on a table is a skull 2202 that a user, such as a surgeon, can see regularly. Then, with the use of the navigation system hardware, through a display with the bar attachment (FIGS. 10-12) or through the navigation system headset (e.g., headset 1506 of FIG. 15), the user can see an overlaid image of a slice of what could have been inside in the skull 2202, using previously recorded image data. Here, the data includes a cross section of the brain and internal passageways that may have been obtained through magnetic resonance imaging. In addition, the navigation system (e.g., system 300 of FIG. 3) of the present disclosure is capable of overlaying even more imaging datasets together at the same time. For example, X-ray data of the skull 2202 could also be superimposed along with the MR data. Rather than the user conventionally seeing the different views of the head in these three different views side by side, the navigation system (e.g., system 300 of FIG. 3) of the present disclosure allows for a user to see how they all smoothly relate by being superimposed onto each other at the precise locations of where they would be.

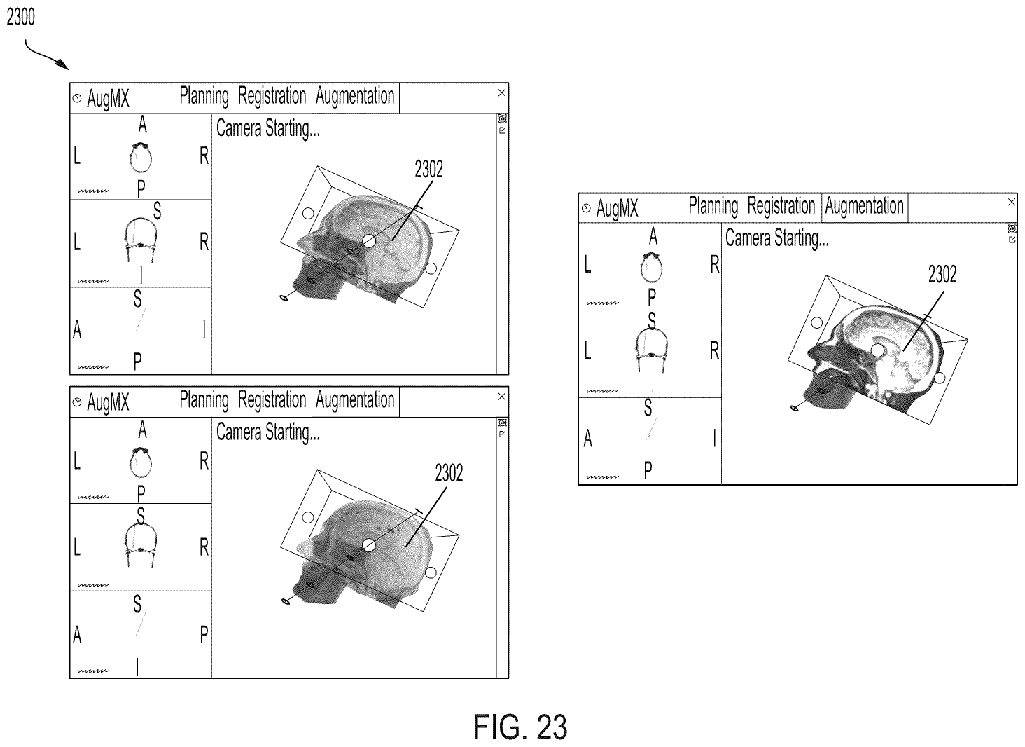

FIG. 23 shows examples of various degrees of opacity 2300 of one of the sets of image data superimposed on the skull 2302 that is regularly in view, according to some embodiments. As shown, the clarity of one set of views can be increased or decreased, as desired, using the software of the present disclosure.

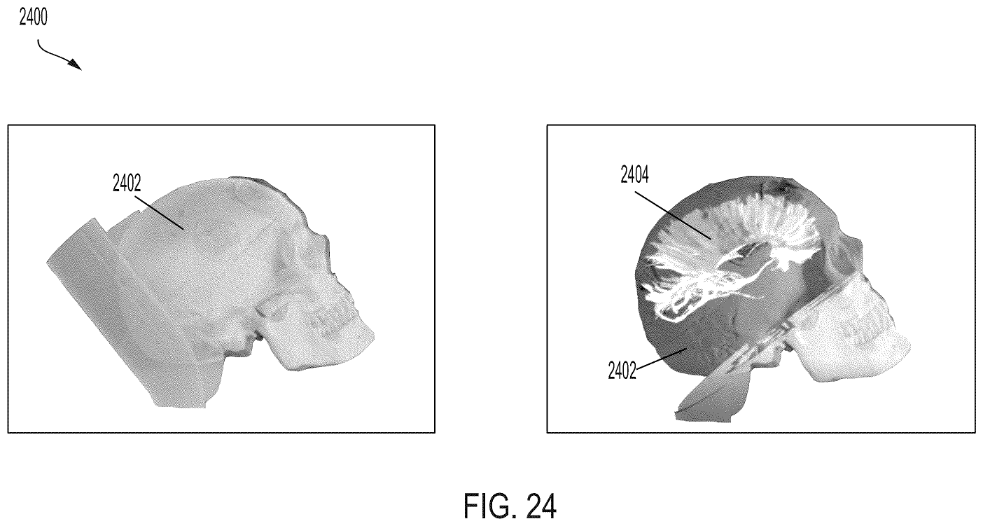

FIG. 24 provides another example of the navigation system providing multiple overlays 2400, according to some embodiments. In this example, a patient is in an operating room and elevated. The patient's head 2402 is resting on a support, as shown on the left. The rest of the patient is covered. A surgeon using the navigation system of the present disclosure may use imaging data of the patient's skull to be superimposed over the live view of the patient's head, as shown on the left. In addition, the surgeon may also superimpose just a portion of imaging data of a section of the patient's brain 2404, onto the same view, as shown on the right. The location of the specified brain matter 2404 is placed in precisely the location of where it resides inside the patient's head 2402, so that the surgeon can see how the position of the patient's skull 2402 is in relation to a desired portion of the patient's brain 2404. As discussed in the software section above, these various co-registered sets of data may be first obtained from fixed imaging techniques, like from an MRI and an X-ray scan. Even though the scans are obtained in 2D slices, various 3D software imaging techniques can be performed preliminarily to generate a 3D rendering of the 2D image data. Then, the 3D rendering of the image data can be superimposed in the correct position to the regular view of the patient, and the surgeon will be able to view all of the sets of data from different angles as the surgeon moves around the patient.

FIGS. 25 and 26 provide example fixed markers 2500, 2600 that provide universal references points to enable the multiple sets of image data to be superimposed onto the patient, according to some embodiments In FIG. 25, shown is a device with four markers 2502 arranged non-symmetrically, which can be placed in a constant position near the target patient. The software may look for these four points 2502 as visual cues to orient the images correctly, based on referring back to these same four points 2502 in other sets of image data. As another example, shown in FIG. 26 is an instrument 2600 that may be attached to the patient or onto a fixed position of the operating table also having four points 2602 as fixed visual cues. These are referred to by the navigation software to calibrate where the AR images should be placed.

In some embodiments, the navigation software of the present disclosure may rely on unique features in the image data and/or in the real-time view of the user, e.g., surgeon, to find a fixed reference point. For example, the navigation software may identify the patient's eyes or eye sockets as reference points relative to the patient's skull. These kinds of cues may be useful when portions of the patient are covered, and maintaining view of the artificially placed reference markers is not always a guarantee. Similarly, the types of reference points on or near the patient can be changed as the software is continually processing the moving surgeon.

As shown in the examples of FIGS. 22, 23, and 24, the navigation system (e.g., system 300 of FIG. 3) of present disclosure is capable of overlaying digital images onto a live image in real time, and fixing the digital images to the same position of the live object even as the viewer moves around the object in real time. This may be referred to as a fusion process, whereby the navigation system hardware, such as the headgear (e.g., headset 1506 of FIG. 15) or a mobile computer including the bar attachment (FIGS. 10-12), performs the fusing process in real time. Consistent with the software algorithms described in FIGS. 16-21, particularly FIG. 17, the navigation system (e.g., system 300 of FIG. 3) may first receive digital content related to the object, such as 3D renderings of combined slices of MR scans or CT scans. The navigation system (e.g., system 300 of FIG. 3) may perform a 3D fusing technique that includes matching the shape of the digital images with what is seen of the live object in real time. As an example, the navigation system (e.g., system 300 of FIG. 3) may view a patient's head in real time, while the navigation system (e.g., system 300 of FIG. 3) accesses x-ray data of the patient's skull and MR data of the patient's brain. One or more transformations may need to be performed by the software to correctly size the digital content with the size of the patient's head as currently viewed.

In some cases, the navigation system (e.g., system 300 of FIG. 3) software may also perform a 2D fusing process of one or more of the digital images. The navigation system (e.g., system 300 of FIG. 3) software may accomplish this by performing one or more rotations of the 2D images to match the angle of the live object. The navigation system (e.g., system 300 of FIG. 3) software may then display an overlay of one or both of 3D and 2D images over the live object, and may keep track of the angle and position of the viewer of the live object in order to continually keep proper orientation of the 3D and 2D images while the viewer moves around the object. As previously discussed, unique reference markers for each object desired to be fused may be used for the navigation system to identify what is the current angle and position of the object relative to its field of view. Examples of these markers 2502, 2602 are shown in FIGS. 25 and 26. As previously mentioned, the navigation system (e.g., system 300 of FIG. 3) of the present disclosure may be capable of fusing these digital images to a real-time live object, with accurate orientation as the viewer moves around the real-time live object, to within an accuracy of placement of 0.1 mm.

In some embodiments, the reference markers (e.g., markers 2502, 2602 of FIGS. 25 and 26) are also included on the surgical or medical instruments that are involved in a medical procedure of the patient. This can allow for the navigation system (e.g., system 300 of FIG. 3) to incorporate the movements of the medical device and provide an augmented reality interaction of the medical device with the live object and the overlays, using the techniques described here. In this way, a remote user may be able to show how a medical device can or should interact with the patient and relevant parts inside the patient, even though the remote user is physically away from the patient. These techniques can also be used for practicing or preparing from a remote location. As such, the disclosures herein can provide a powerful tool for improving preparation of a medical procedure, either by providing practice with an accurate replica of patient data, and/or by providing a teaching tool to train others.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

References to “or” may be construed as inclusive so that any terms described using “or” may indicate any of a single, more than one, and all of the described terms. The labels “first,” “second,” “third,” and so forth are not necessarily meant to indicate an ordering and are generally used merely to distinguish between like or similar items or elements.

Various modifications to the implementations described in this disclosure may be readily apparent to those skilled in the art, and the generic principles defined herein may be applied to other implementations without departing from the spirit or scope of this disclosure. Thus, the claims are not intended to be limited to the implementations shown herein, but are to be accorded the widest scope consistent with this disclosure, the principles and the novel features disclosed herein.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or any suitable combination thereof), registers, or other machine components that receive, store, transmit, or display information. Furthermore, unless specifically stated otherwise, the terms “a” or “an” are herein used, as is common in patent documents, to include one or more than one instance. Finally, as used herein, the conjunction “or” refers to a non-exclusive “or,” unless specifically stated otherwise.

The present disclosure is illustrative and not limiting. Further modifications will be apparent to one skilled in the art in light of this disclosure and are intended to fall within the scope of the appended claims.

1. An augmented reality device comprising:
a housing;
a depth camera coupled to the housing and configured to provide image data with a 3-dimensional component;
a visual camera coupled to the housing and configured to provide extra-sensory image data that a human user cannot see naturally; and
an overlay display component configured to receive at least two sets of image data and overlay both of the at least two sets of image data onto a common point of reference in a user's field of view.

2. The augmented reality device of claim 1, further comprising a headset configured to support the housing.

3. The augmented reality device of claim 2, wherein the depth camera and the visual camera are positioned on the headset such that the user's field of view coincides with the both the fields of view of the depth camera and the visual camera.

4. The augmented reality device of claim 2, wherein the overly display component is positioned over the user's field of view as the user wears the headset.

5. The augmented reality device of claim 1, further comprising a bar attachment configured to attach to a mobile device.

6. The augmented reality device of claim 5, wherein the overlay display component utilizes a visual display of the mobile device.

7. A system for surgical navigation, the system comprising:
a first augmented reality (AR) device positioned in a local geographic location;
a second augmented reality device positioned in a remote geographic location and wired or wirelessly coupled to the first AR device; and
a software system coupled to both the first AR device and the second AR device and configured to:
process real-time image data produced by the first AR device;
access fixed medical image data recorded previously; and
cause the second AR device to display the real-time image data and the fixed medical image data superimposed over the real-time image data.

8. The system of claim 7, wherein the first AR device is configured to identify a fixed reference marker in the field of view and transmit image data about the fixed reference marker to the second AR device.

9. The system of claim 8, wherein the software system is configured to orient the fixed medical image data to the real-time image data using the image data about the fixed reference marker.

10. The system of claim 7, wherein the fixed medical image data comprises 2D and 3D image data.

11. The system of claim 7, wherein the software system is configured to cause display of both the 2D and 3D image data superimposed over the real-time image data, simultaneously.

12. The system of claim 7, wherein the superimposed 2D and 3D data over the real-time image data represents one or more views of physical content within or inside an object of the real-time image data.

13. A method of augmented reality (AR) for fusing digital image data of an object to a real-time view of the object, the method comprising:
accessing, in real-time, a view of the object;
accessing the digital image data of the object, the digital image data of the object previously captured and stored as one or more static digital images of the object; and
performing a fusion technique that affixes the digital image data to the view of the object in real-time, using an augmented reality display screen, such that the digital image data stays affixed to the view of the object in real-time as the view of the object changes in position or orientation within the augmented reality display screen.

14. The method of claim 13, wherein the digital image data comprises 3D digital image data of the object.

15. The method of claim 13, wherein the digital image data comprises 2D digital image data of the object.

16. The method of claim 13, further comprising:
accessing 2D digital image data of the object; and
performing a 3D rendering technique to transform the 2D digital image data into 3D digital image data of the object; and
wherein the fusion technique comprises affixing the 3D digital image data of the object to the view of the object in real-time.

17. The method of claim 14, wherein the fusion technique comprises matching a size of the view of the object in real-time with a size of the 3D digital image data, such that the size of the 3D digital image data is displayed in correct proportion with the size of the object.

18. The method of claim 14, wherein the fusion technique comprises matching a shape of the view of the object in real-time with a shape of the 3D digital image data, such that the shape of the 3D digital image data is displayed in correct proportion with the shape of the object.

19. The method of claim 13, further comprising accessing a fixed reference marker near the view of the object in real-time, wherein the fixed reference marker provides sufficient data to provide a unique 3 dimensional orientation, and depth, of the view of the object, even as the position or orientation of the view of the object changes.

20. The method of claim 19, wherein performing the fusing technique comprises utilizing the fixed reference marker to affix the digital image data to the view of the object in real-time.
